(19) United States
(12) Patent Application Publication
(10) Pub. No.: US 2007/0214609 A1
(43) Pub. Date: Sep. 20, 2007

(54) A method and an installation for determining characteristics representive of a physical and/or chemical transformation occuring in a microreactor

(76) Inventors: Pascal Pitiot, Lyon (FR); Francois Metz, Irigny (FR); Serge Henrot, Sainte-Catherine (FR)

Correspondence Address: Ralph A. Dowell of DOWELL & DOWELL P.C., 2111 Eisenhower Ave, Suite 406, Alexandria, VA, 22314, US

(21) Appl. No.: 10/584,210

(22) PCT Filed: Dec. 22, 2004
(86) PCT No.: PCT/FR04/03356

§ 371 Date: May 16, 2007

(30) Foreign Application Priority Data
Dec. 24, 2003 (FR) 0315396

(51) Int. Cl. B01D 9/00; G01N 33/00

(52) U.S. Cl. 23/295 R; 73/866

(57) ABSTRACT

The method comprises the steps of: establishing a flow of the medium under steady conditions through at least one region (6) of the microreactor; using analyzer means (11) to access the steady flow at least one point (6₁, 6₂); measuring at least one magnitude characteristic of the medium at the or each point (6₁, 6₂) by using the analyzer means (11); and determining (via 10′; BR) characteristics representative of the transformation as a function of the result of the or each measurement.

The present invention relates to a method and an installation for determining characteristics representative of a physical and/or chemical transformation occurring in a microreactor.

The term “transformation” is used to mean in particular a reaction of chemical and/or physical type, such as, for example, any type of conventional chemical reaction, and likewise crystallization or precipitation, or indeed a modification to a liquid/vapor equilibrium, etc.

In the meaning of the invention, characteristics representative of the transformation can initially be determined by determining the parameters specific to the transformation. These parameters relate in particular to kinetic, thermodynamic, or other parameters. Determining these parameters is highly advantageous insofar as they provide in-depth knowledge about the transformation in question.

In the meaning of the invention, characteristics representative of the transformation can also be determined by determining parameters concerning the running of the transformation in the context of the method on a pilot scale or on an industrial scale, and in which the above-mentioned transformation occurs. These running parameters are in particular changes applied to temperature, to flow rate, and to input concentrations of substances involved during the transformation.

Microreactors are tools used in particular in the fields of analytic chemistry, biochemistry, clinical diagnosis, medical chemistry, and the chemical industry. The characteristic dimension of microreactors to which the invention applies lies in the range about 10 micrometers (μm) to about 1 millimeter (mm). A typical microreactor is described for example in EP-A-0 616 218.

It is already known to use a microreactor to determine parameters specific to a transformation, as mentioned above. However, in conventional manner, the reaction stream is analyzed solely at the outlet from the microreactor, either when the transformation has been completed or has reached a desired state of advance, or else by stopping advance of the transformation, by quenching or by analogous means.

That known solution nevertheless presents certain drawbacks.

It requires numerous measurements and numerous tests to be performed, and as a result it requires numerous stops (quenches or other stops) of the transformation. That solution thus implies considerable time for carrying out an investigation.

Furthermore, it does not necessarily guarantee accuracy that is sufficient for the analysis carried out in that way. When quenching or intrusive analyzer means are used, such lack of accuracy can be due in particular to the fact that the volume of the analysis cell is of the same order or even greater than the volume of the microreactor itself. Under those circumstances, the transformation runs the risk of continuing within the analysis volume, even when using quenching means.

That said, the present invention seeks to remedy those various drawbacks.

To this end, the invention provides a method of determining characteristics representative of a physical and/or chemical transformation, in particular a reaction, the transformation occurring in a medium, in particular a reaction medium, flowing within at least one microreactor, the method comprising the following steps:
establishing a flow of the medium under steady conditions through at least one region of the microreactor;
using analyzer means to access the steady flow at at least one point;
measuring at least one magnitude characteristic of the medium at the or each point by using the analyzer means; and
determining characteristics representative of the transformation as a function of the result of the or each measurement.

In the meaning of the invention, the or each magnitude characteristic of the medium, as measured by the analyzer means, is for example the concentration in one and/or the other of the reagents, reactants, and/or substances involved within the transformation, or indeed temperature or density.

Steady conditions can be defined, in conventional manner, as conditions in which the following are substantially constant over time: firstly the various magnitudes of the transformation involved in the medium at a given point thereof, and secondly the various parameters relating to the flow of the medium, such as, in particular, its flow rate. The person skilled in the art knows how to set up such a steady flow in the microreactor, in conventional manner.

In the meaning of the invention, an analyzer means is the active element of an analysis appliance that extends between the body of said appliance and the medium for analysis. Thus, one such analyzer means may be a laser beam for a Raman spectrum, an ultraviolet beam or an infrared beam for a spectrometer, or indeed a temperature probe, an appliance for determining density in line, or more simply visual inspection.

The invention makes it possible in particular to achieve the above-mentioned objects.

It makes it possible to follow a reaction “in situ”, i.e. within the microreactor itself, as opposed to following a reaction at the outlet from the microreactor, as in the prior art. Under such circumstances, characteristics representative of the transformation are determined by the invention with accuracy that is considerably increased compared with that prior art.

In addition, the invention makes it possible to determine all of the selected chemical and/or physical parameters, while implementing a single transformation, without there being any need to repeat the same transformation several times by implementing successive quenching operations or tests at varying times of passage.

It should be observed that the way characteristics of a transformation are determined in standard reactors cannot easily be transposed to the microreactors to which the invention applies. Thus, a reactor, even when small in size, cannot be considered as being a microreactor, given that those two types of tool present specific features that are very different.

The above-mentioned differences between microreactors and reactors of standard type are particularly significant on a pilot scale or on an industrial scale. Those two types of reactor are accompanied by extrapolations that are totally different, or even opposite. Thus, with standard type reactors, an extrapolation that is used involves changing the size of the reactor.

This should be compared with replication, which is the extrapolation used in the field of microreactors, where replication consists in placing a plurality of such microreactors in parallel, without significantly changing their dimensions. In this respect, microreactors make it easier to investigate transformations presenting kinetics that are very fast, since they enable transformations to be performed under high or very high pressures with smaller risks of explosion. They also present great resistance to high temperatures, which serves to reduce any risk of thermal runaway.

Because of their size, microreactors are also very advantageous from an economic point of view and from the point of view of the toxicity of the various substances of the transformation implemented. The small quantities of said substances that are used, ensure that such microreactors are tools that are very safe and that present high performance compared with standard reactors.

Furthermore, the method implemented in the state of the art by means of microfluidic type systems likewise cannot be transposed in simple manner to the fields to which the present invention applies, and for the same reasons as those mentioned above.

Specifically, in the field of microfluidics, there are problems of heat transfer, and insufficiencies in terms of being able to withstand pressure, which can be remedied by microreactors. Microreactors are thus much more versatile than microfluidic systems, while still possessing very small size.

It should also be observed that the invention differs clearly from a method in which the parameters of a transformation within the microreactor are merely verified, i.e. where such parameters have already been determined beforehand. In the invention, the flow of the medium through the microreactor serves not to perform a validation step, but to perform an additional step of determining characteristics, thus giving access to characteristics that are not known a priori.

According to an advantageous characteristic of the invention, the steady flow is accessed at different points that are distinct from one another in time and/or space. This provides knowledge in greater depth and more quickly about the transformation for which it is desired to determine the representative characteristics.

In a first variant of the invention, different points are accessed that are distinct from one another in space. In other words, when implementing the method of the invention, relative movement is involved between the analyzer means and the steady flow of the medium.

In order to implement this first variant, it is possible firstly to move the microreactor while keeping the analyzer means stationary. Alternatively, it is also possible to move the analyzer means while keeping the microreactor stationary.

It is also possible to perform a plurality of measurements at a single point, the measurements being distinct from one another in time, while maintaining the analyzer means and the medium in which the transformation occurs immobile relative to each other. When the flow begins under transient conditions, this makes it possible to gain access to large amounts of information relating to transient conditions before steady conditions become established.

According to an advantageous characteristic of the invention, the analyzer means is non-destructive with respect to the medium in which the transformation takes place. This avoids any interaction, in particular of the physico-chemical type, between the analyzer means and the medium, which might spoil the quality of the parameters.

In a first implementation, the analyzer means is invasive. This means that it penetrates physically through at least one wall of the microreactor. Under such circumstances, it might be a temperature sensor, for example.

In another implementation, access to the flow under steady conditions is obtained through a zone of the microreactor that is permeable to the analyzer means. In other words, the analyzer means is capable of going through the said zone, without spoiling its own characteristics.

The permeable zone may form substantially the entire body of the microreactor, or in a variant it may be fitted thereto. When fitted, it may be constituted, for example, by a window that is secured to the body of the microreactor, in particular by brazing.

It will be understood that the nature of the permeable zone varies depending on the specific nature of the analyzer means. Thus, the zone may be permeable to waves, in particular it may be permeable to visible radiation, to ultraviolet radiation, or indeed to any electromagnetic radiation.

It is recalled that the transformation having parameters that are to be determined by means of the invention is constituted in particular by a reaction, e.g. of the chemical type, and/or of the physical type, or indeed a crystallization.

According to another characteristic of the invention, the steady flow rate lies in the range 1 milliliter per hour (mL/h) to 1 liter per hour (L/h), and preferably lies in the range 0.1 L/h to 1 L/h.

In a first implementation of the invention, parameters specific to the transformation are determined as characteristics representative of said transformation. As mentioned above, it is recalled that such parameters are, for example, the concentration of one and/or the other of the reagents, reactants, and/or substances involved within the transformation, or indeed temperature or density.

In a second implementation of the invention, running parameters of the transformation are determined as characteristics representative of the transformation. Such running parameters are constituted in particular by changes applied to the temperature, to the flow rate, and to the inlet concentration of the substances involved during the transformation.

In this second implementation, and advantageously, the or each microreactor within which the running parameters of the transformation are determined is/are disposed in parallel with other microreactors, and the various microreactors are fed with the same media, possessing the same flow rates, and under the same operating conditions.

As a result, the various microreactors form a single reactor capable of presenting a pilot scale or even an industrial scale. Furthermore, it should be observed that the other microreactors are of conventional type, i.e. in particular, they are not provided with means for accessing the flow under steady conditions.

According to an advantageous characteristic of the invention, the various parallel-connected microreactors are fed by means of a single upstream feed line.

According to another advantageous characteristic, at least one instantaneous value is obtained of at least one magnitude characteristic of the medium, the or each instantaneous value is compared with a reference value for the or each characteristic magnitude, and the running of the transformation is modified as a function of the value of the ratio between said measured value and said reference value.

The invention also provides an installation for determining characteristics representative of a physical and/or chemical transformation, in particular a reaction, for implementing the method in accordance with any preceding claim, said transformation occurring in a medium, in particular a reaction medium, and the installation comprising:
at least a first microreactor through which said medium is suitable for flowing;
an analyzer means;
means for accessing at least one point of a flow of the medium under steady conditions in at least one region of the first microreactor;
means for taking at least one measurement of at least one magnitude characteristic of the medium in the or each point; and
means for determining characteristics representative of the transformation as a function of the result of the or each measurement.

According to other characteristics of the invention:
displacement means are provided suitable for displacing the analyzer means and the microreactor relative to each other;
the analyzer means is non-destructive relative to the reaction medium;
the analyzer means is intrusive, in particular the sensor is a probe;
the access means comprise a zone of the microreactor that is permeable to the analyzer means, in particular a window that is transparent to visible light;
the means for determining characteristics representative of the transformation are means for determining parameters specific to said transformation;
the means for determining parameters specific to said transformation include a computer;
the means for determining characteristics representative of the transformation are means for determining running parameters for said transformation;
the means for determining running parameters of the transformation comprise a regulation loop;
the regulation loop possess a measurement line put into communication with the analyzer means and suitable for providing at least one instantaneous value of at least one characteristic magnitude, a reference line suitable for providing at least one reference value for at least one characteristic magnitude, and an output line put into communication with means for running the reactor;
the installation further comprises at least one other microreactor connected in parallel with the or each first microreactor; and
the various microreactors are fed by means of a single upstream feed line.

The invention is described below with reference to the accompanying drawings given purely by way of non-limiting example, and in which:

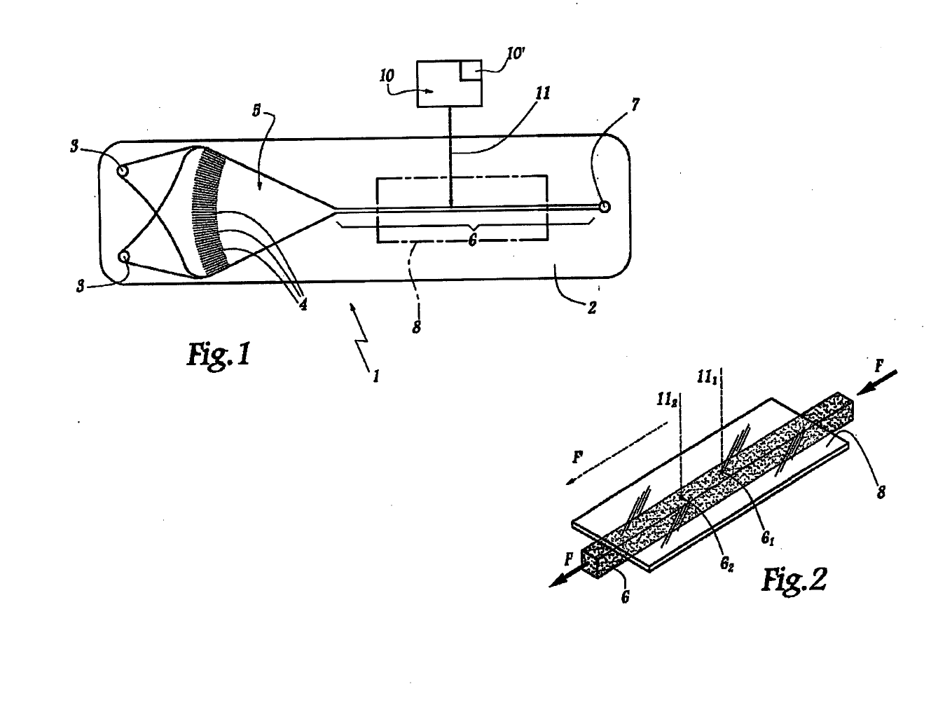

FIG. 1 is a diagrammatic face view showing the various elements of an installation in accordance with the invention;

FIG. 2 is a perspective view on a larger scale showing the implementation of the method in accordance with the invention in a specific region of the FIG. 1 installation; and

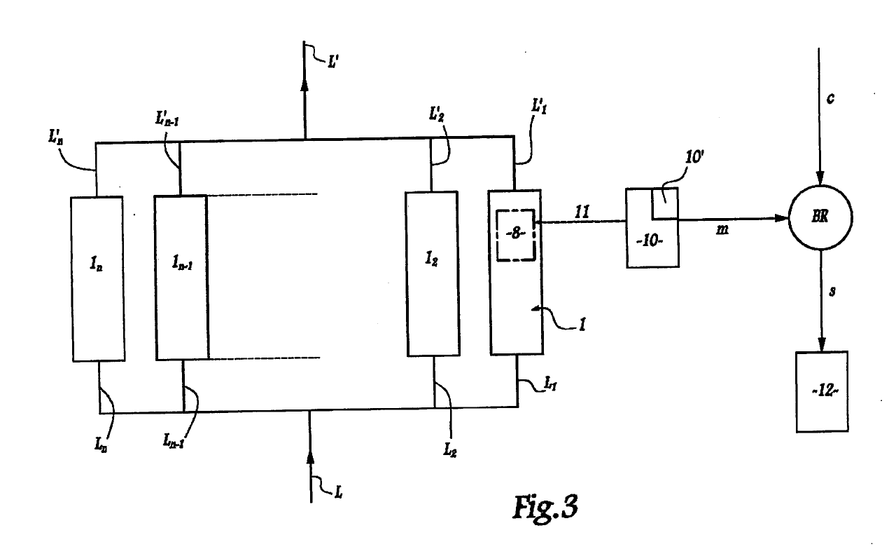

FIG. 3 is a diagrammatic face view analogous to FIG. 1 showing the various elements of an installation in accordance with a variant embodiment of the invention.

FIG. 1 shows a microreactor given overall reference 1. It comprises a body 2, e.g. made of metal or stainless steel, having formed therein in conventional manner two inlets 3 into which two different reagents can be introduced. Nevertheless, in a variant, there could be some other number of inlets, e.g. lying in the range 1 to 10, and preferably 2 or 3.

Downstream from these inlets, there are formed a plurality of upstream channels 4 in parallel. As an indication, there may be provided 124 of these channels, for example, each having a cross-section of 0.005 square millimeters (mm²), for example.

Nevertheless, in a variant, it is possible to provide some other number of channels, e.g. a number lying in the range 1 to 10,000, advantageously in the range 10 to 1,000, and of cross-section that is different from that of the above example.

Downstream from these upstream channels 4, there extends a constriction zone 5 which opens out into a “main” downstream channel 6 having a length of 40 millimeters (mm) and of section equal to 0.25 mm², for example.

In a variant, the channel 6 could have a length different from that mentioned above, for example lying in the range 1 mm to 1 meter (m), and preferably lying in the range 15 mm to 50 mm, and it could also have a section that is different from that mentioned above. In addition, the channel 6, which is shown as being rectilinear in shape, could likewise present some other profile, for example it could be sinusoidal.

In a variant, provision can be made to dissociate the upstream channel 4 physically from the downstream channel 6. Under such circumstances, the various channels 4 are made, for example, within a first plate which can be secured in releasable manner relative to another plate having the main channel 6 formed therein.

Returning to FIG. 1, the main channel 6 opens out into an outlet 7, e.g. connected to a conventional effluent treatment system. The microreactor 1 also has a cover that is not shown, in which there is included a transparent window 8, the cover being secured by any appropriate fastener means. Once the cover covers the body 2, the window 8 overlies at least a portion of the main channel 6. For reasons of clarity, the outline of the window 8 is drawn in chain-dotted lines in FIG. 1.

Provision can also be made for means (not shown), e.g. electrical or pneumatic means, to drive the reagents in conventional manner from the inlets 3 towards the outlet 7 via the channels 4, the constriction 5, and the main channel 6. The installation shown in FIG. 1 further comprises an analyzer appliance 10, specifically in this example of the Raman type. In operation, this analyzer 10 uses a laser beam 11 that constitutes an analyzer means.

A non-limiting implementation of the method of the invention is described below with reference to FIGS. 1 and 2.

Two reagents, labeled respectively A and B, are introduced continuously into the inlets 3 and flow along the channels 4 and then progressively into the constriction 5 before flowing into the main channel 6, along arrows F in FIG. 2. It should be observed that the steps described immediately above serve to mix the reagents A and B together in very intimate manner.

In a variant, an arrangement other than that described above could be provided, providing it ensures that the reagents are well mixed together at least starting from the first measurement point, e.g. at the inlet to the main channel 6. For this purpose, the various channels of the microreactor may in particular be T-shaped, as is known per se.

Returning to the present implementation, it is assumed that the mixture of A and B constitutes a medium, specifically a reaction medium, that is liable to be subject to a transformation, specifically a chemical reaction. The products of this reaction are referenced C and D.

When the flow of the mixture formed by A and B reaches steady conditions, a beam 11 is directed to a first point 61 of the reaction medium. For reasons of clarity, the reference 11₁ is given to the position P of said beam 11 at a residence time tₛ of the medium.

In this position 11₁, the beam 11 then produces a measurement of at least one magnitude representative of the reaction medium. For example this could be the concentrations of the reagents [A]₁ and [B]₁ and also of the reaction products [C]₁ and [D]₁, or indeed the temperature or the density of the reaction medium.

Once the above measurement has been made, the beam 11 is moved along the channel 6 in its downstream direction along arrow F′. The beam is then directed to another point of the reaction medium, referenced 6₂, corresponding to a position P+δP of the beam, referenced 11₂, relating to a residence time tₛ+δtₛ for the reaction medium.

In its second position 11₂, the beam 11 then performs a second measurement of at least one representative magnitude of the reaction medium, in a manner analogous to that described with reference to the first position 11₁. For example the magnitude may be the concentrations [A]₁, [B]₁, [C]₁, and [D]₁. Then, in a manner that is not shown, the beam 11 continues to be displaced towards the downstream end of the channel 6 so as to take a series of measurements of at least one magnitude representative of the reaction medium.

At the end of this series of measurements, knowledge of the various magnitudes gives access, in known manner, to various parameters of the reaction. This determination can be implemented, for example, by a computer 10′ integrated in the analyzer 10.

Finally, it should be observed that it is also possible to envisage not displacing the beam 11 relative to the microreactor 1 as a function of time. Under such conditions, the laser beam 11 serves to take different measurements at points that are distinct from one another no longer in space, but in time. This can be used in particular to verify the reproducibility of measurements, and consequently to ensure that conditions are indeed steady.

FIG. 3 shows a variant embodiment of the invention.

The microreactor 1 associated with the analysis appliance 10 is integrated within an installation that comprises (n−1) other microreactors, given references 12 to 1ₙ. It should be observed that the other microreactors are generally identical to the microreactor referenced 1. However they do not have respective zones permeable to the analyzer means, such as the transparent window 8 shown in FIG. 1.

These n microreactors 1 to in are fed from a main upstream line L which is subdivided into n secondary upstream lines referenced L₁ to Lₙ. Downstream from the microreactors, there are provided secondary downstream liens L′₁ to L′ₙ which are grouped together to form a single main downstream line L′.

It should be observed that the reaction medium possesses a flow rate referenced Q in the main lines L and L′. Otherwise, in each of the secondary lines respectively L₁ to Lₙ and L′₁ to L′ₙ, the medium possesses the same flow rate, namely Q/n.

It should be observed that the installation shown in FIG. 3 constitutes a single reactor suitable for presenting a pilot scale or an industrial scale, formed by replicating the microreactors which may be provided in very large numbers, for example about 100. In this respect, although the flow rate Q/n within each microreactor is relatively small, the overall flow rate Q may present a value that is high, since a very large number of microreactors can be placed in parallel.

In operation, the various transformations that take place within the microreactors 1 to in are all identical, both concerning their nature and their state of advance. The various microreactors are fed with the same substances, at the same flow rates, and they are placed in the same operating conditions.

A series of measurements are then taken of magnitudes representative of the reaction medium flowing through the reactor 1. This operation is undertaken in a manner analogous to that described with reference to FIGS. 1 and 2.

These representative magnitudes are said to be instantaneous and constitutes the measurement m within a regulation loop, referenced BR. The reference value c for said regulation loop BR is constituted by reference values for the above-mentioned magnitudes representative of the transformation.

Finally, the output s from the regulation loop is applied to apparatus given overall reference 12. This apparatus serves to modify the general parameters under which the method is run, thereby enabling the transformation to be implemented.

It should be observed that in the embodiment of FIG. 3, the parameters specific to the transformation itself are not determined by the analysis appliance itself since they are already known beforehand. The analysis appliance thus serves at all times to compare the instantaneous magnitudes characteristic of the medium in which the transformation is taking place with reference values. Where appropriate, this makes it possible in real time to modify the general parameters of the overall reactor as constituted by the various microreactors in parallel, so as to cause the instantaneous magnitudes to come closer to the predefined reference values.

In a variant that is not shown, measurements of the kind performed on the microreactor 1 can be taken from a plurality of the microreactors. The different measured instantaneous values are then compared with one another, e.g. in order to obtain a mean value which is then compared with the reference value. This makes it possible to verify that the various microreactors are working properly, and consequently that the inlet flow is indeed taking place in parallel.

1. A method of determining characteristics representative of a physical and/or chemical transformation, in particular a reaction, the transformation occurring in a medium, in particular a reaction medium, flowing within at least one microreactor (1), the method comprising the following steps:
establishing a flow of the medium under steady conditions through at least one region (6) of the microreactor;
using analyzer means (11) to access the steady flow at least one point (6₁, 6₂);
measuring at least one magnitude characteristic of the medium at the or each point (6₁, 6₂) by using the analyzer means (11); and
determining (via 10′; BR) characteristics representative of the transformation as a function of the result of the or each measurement.

2. A method according to claim 1, characterized in that the steady flow is accessed at different points (6₁, 6₂) that are distinct from one another in time and/or space.

3. A method according to claim 2, characterized in that different points (6₁, 6₂) are accessed that are distinct from one another in space.

4. A method according to claim 3, characterized in that, in order to access the different points, the microreactor is displaced while keeping the analyzer means stationary.

5. A method according to claim 3, characterized in that, in order to access the different points, the analyzer means is displaced while keeping the microreactor stationary.

6. A method according to claim 1, characterized in that the analyzer means is non-destructive with respect to the reaction medium.

7. A method according to claim 1, characterized in that the analyzer means is invasive, in particular the sensor is a probe.

8. A method according to claim 1, characterized in that the or each point of the steady flow is accessed through a zone (8) of the microreactor (1) that is permeable to the analyzer means (11), in particular a window (8) that is transparent to visible light.

9. A method according to claim 1, characterized in that the transformation is a chemical and/or physical reaction.

10. A method according to claim 1, characterized in that the transformation is a crystallization.

11. A method according to claim 1, characterized in that the steady flow possesses a rate lying in the range 1 mL/h to 1 L/h, and preferably in the range 0.1 L/h to 1 L/h.

12. A method according to claim 1, characterized in that parameters specific to the transformation are determined (by 10′) as characteristics representative of said transformation.

13. A method according to claim 1, characterized in that running parameters of the transformation are determined (by BR) as characteristics representative of the transformation.

14. A method according to claim 13, characterized in that the or each microreactor (1) within which the running parameters of the transformation are determined is/are disposed in parallel with other microreactors (1₂, . . . , 1ₙ), and the various microreactors are fed with the same media, possessing the same flow rates, and under the same operating conditions.

15. A method according to claim 14, characterized in that the various parallel-connected microreactors (1, 1₂, . . . 1ₙ) are fed by means of a single upstream feed line (L).

16. A method according to claim 13, characterized in that at least one instantaneous value (m) is obtained of at least one magnitude characteristic of the medium, the or each instantaneous value is compared with a reference value (c) for the or each characteristic magnitude, and the running of the transformation is modified (by s) as a function of the value of the ratio between said measured value and said reference value.

17. An installation for determining characteristics representative of a physical and/or chemical transformation, in particular a reaction, for implementing the method in accordance with claim 1, said transformation occurring in a medium, in particular a reaction medium, and the installation comprising:
at least a first microreactor (1) through which said medium is suitable for flowing;
an analyzer means (11);
means (8) for accessing at least one point of a flow of the medium under steady conditions in at least one region (6) of the first microreactor;
means (10, 11) for taking at least one measurement of at least one magnitude characteristic of the medium in the or each point; and
means (10′; BR) for determining characteristics representative of the transformation as a function of the result of the or each measurement.

18. An installation according to claim 17, characterized in that displacement means are provided suitable for displacing the analyzer means (11) and the microreactor (1) relative to each other.

19. An installation according to claim 17, characterized in that the analyzer means is non-destructive relative to the reaction medium.

20. An installation according to claim 17, characterized in that the analyzer means is intrusive, in particular the sensor is a probe.

21. An installation according to claim 17, characterized in that the access means comprise a zone (8) of the microreactor (1) that is permeable to the analyzer means (11), in particular a window (8) that is transparent to visible light.

22. An installation according to claim 17, for implementing the method according to claim 12, the installation being characterized in that the means for determining characteristics representative of the transformation are means (10′) for determining parameters specific to said transformation.

23. An installation according to claim 22, characterized in that the means for determining parameters specific to said transformation include a computer (10′).

24. An installation according to claim 17, the installation being characterized in that the means for determining characteristics representative of the transformation are means (BR) for determining running parameters for said transformation.

25. An installation according to claim 24, characterized in that the means for determining running parameters of the transformation comprise a regulation loop (BR).

26. An installation according to claim 25, characterized in that the regulation loop (BR) possess a measurement line (m) put into communication with the analyzer means (11) and suitable for providing at least one instantaneous value of at least one characteristic magnitude, a reference line (c) suitable for providing at least one reference value for at least one characteristic magnitude, and an output line (s) put into communication with means (12) for running the reactor.

27. An installation according to claim 24, characterized in that it further comprises at least one other microreactor (1₂, . . . , Iₙ) connected in parallel with the or each first microreactor (1).

28. An installation according to claim 27, characterized in that the various microreactors (1, 1₂, . . . , 1ₙ) are fed by means of a single upstream feed line (L).
